SEQUENCE LISTING (1 sequence)

SEQ ID NO: 1 - protein, 373 aa, homo sapien
RSLLSPTFTSGKLKEMVPIIAQYGDDVLVRNLRREAETGKPVTLKDVFGAYSMDVITSTSFGVNIDSLNNPQDPFVENTKKLLRFDFLDPFFLSIIFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGWVVMIPSYALHRDPKYTEPEKFLPERFSKKNKDNIDPYTYTPFGSGPRNCIGMRFALMNMKLALIRVLQNFSFKPCKETQIPLKLSLGGLLQPEKPVVLKVESRDGTVSGA

(19) United States
(12) Patent Application Publication
(10) Pub. No.: US 2014/0037574 A1
(43) Pub. Date: Feb. 6, 2014

(54) PHARMACEUTICAL COMPOSITION

(71) Applicants: Pius Sedowhe Fasinu, Western Cape (ZA); Viness Pillay, Benmore Sandton (ZA); Yahya Essop Choonara, Lenasia (ZA); Lisa Claire Du Toit, Fleurhof Florida Roodepoort (ZA)

(72) Inventors: Pius Sedowhe Fasinu, Western Cape (ZA); Viness Pillay, Benmore Sandton (ZA); Yahya Essop Choonara, Lenasia (ZA); Lisa Claire Du Toit, Fleurhof Florida Roodepoort (ZA)

(21) Appl. No.: 13/989,153

(22) PCT Filed: Nov. 28, 2011
(86) PCT No.: PCT/IB11/55337

§ 371 Date: Oct. 10, 2013

(30) Foreign Application Priority Data
Nov. 26, 2010 (ZA) 2010/03742

(51) Int. Cl. A61K 31/785; A61K 31/765; A61K 31/78; A61K 31/44

(52) U.S. Cl. CPC A61K 31/785; A61K 31/44; A61K 31/765; A61K 31/78
USPC 424/78.31; 514/356; 424/78.37

(57) ABSTRACT

The invention provides a pharmaceutical composition for oral administration of a pharmaceutically active agent to a subject, including the pharmaceutically active agent and an inhibitor of CYP3A4. Administration of the inhibitor and the pharmaceutically active agent reduces pre-systemic degradation of the pharmaceutically active agent by CYP3A4. The inhibitor can be poly(ethylene glycol), methoxy poly(ethylene glycol), aminated poly(ethylene glycol), O-(2-aminoethyl)-O-methoxy poly(ethylene glycol), polyoxyethylene glycol, branched poly(ethylene glycol), 3-arm poly(ethylene glycol), 4-arm poly(ethylene glycol), 8-arm-poly(ethylene glycol)polyamine, poly(L-lysine), poly(L-arginine), poly(L-alanine), poly(L-valine), poly(L-serine), poly(L-histidine), poly(L-isoleucine), poly(L-leucine), poly(L-glutamic acid), poly(L-glutamine), poly(L-guanidine), poly(methyl methacrylate), polyvinyl acetate, polyacrylate, poly(lactic-co-glycolic acid) and derivatives thereof. A method of treatment is also described.

### FIELD OF THE INVENTION

This invention relates to pharmaceutical compositions which include cytochrome P450 3A4 (CYP3A4) inhibitors for use in inhibiting or reducing inhibition of CYP3A4 metabolism of pharmaceutically active agents which are also in the pharmaceutical compositions.

### BACKGROUND TO THE INVENTION

Research in the field of drug metabolism has attracted more attention in recent times not only because it forms the basis for understanding pharmaco-toxicology but also owing to its growing influence and application in drug delivery and pharmacotherapy [1-4]. Inhibition of first-pass drug metabolism is desirable in order to improve oral drug bioavailability and clinical outcomes [5, 6] while the induction of enzymatic metabolic activities may be applicable in clinical toxicity [7]. The human cytochrome P450 (CYP) enzyme system present in the liver and intestine is responsible for the metabolism of a wide range of xenobiotics (for example drugs, carcinogens and pesticides) and endobiotics (for example prostaglandins, bile acids and steroids) [8-13]. A sub-family of this enzyme system, CYP3A4, is responsible for the metabolism (at least in part) of more than 50% of marketed drugs [14-17]. It is the ability of CYP3A4 to metabolize numerous structurally unrelated compounds that makes CYP3A4 responsible for the poor oral bioavailability of many drugs as they are subjected to pre-systemic CYP3A4-mediated metabolic activity [18, 19]. Pre-systemic metabolism of pharmaceutical compounds occurs when orally administered pharmaceutical compounds are metabolized during their passage to the systemic circulation from the gut lumen. Typical organs that play an important role in pre-systemic metabolism include, for example the liver and the intestine where CYP3A4-mediated metabolic activity is known to occur.

A large number of documented drug-drug and drug-food interactions have also been attributed to intestinal and liver microsomal CYP activity [20-22]. Many drugs are being designed using non-oral delivery means to circumvent the problems associated with oral drug administration. However, oral drug administration is generally accepted as the most preferred route of systemic drug delivery due to convenience, patient compliance and possibility of self-administration. Recently, researchers have shown that approximately 60% of currently marketed drugs are oral products [23]. Greater than 90% of all orally administered drugs are subjected to CYP-mediated pre-systemic metabolism, resulting in a loss of active drug which in certain cases could be as high a 95% of the administered dose [24-26].

This significant loss has both pharmacological and economic implications. The allowances made in oral drug formulations for pre-systemic loss of active ingredients necessitates the use of relatively high doses of drugs. Adverse effects of drugs are generally more pronounced with an increase in dosage. Furthermore, drugs with narrow therapeutic and safety margins and whose absorptions may be erratic and unpredictable portend risks of toxicity and therapeutic failure. On the economic front, patients and consumers bear the increased cost of additional actives incorporated into formulations to account for loss to first-pass drug inactivation.

Pre-systemic enzymatic inhibitors can modulate enzyme systems such as CYP3A4 to enhance oral bioavailability of active pharmaceutical compounds contained in oral pharmaceutical dosage forms. The search for an effective pre-systemic enzyme inhibitor is therefore an important component of the earnestly sought solution to the many challenges of effective oral drug delivery. It is important that an oral bioavailability enhancer should be pharmacologically inert, biocompatible and biodegradable. Such compounds should also exert temporary and/or reversible inhibition on metabolic enzymes to allow systemic drug clearance and prevent accumulation. It is also important that such enhancers should be economically affordable in comparison to the cost of the active pharmaceutical compound.

It has been suggested that reported interactions between grapefruit juice and concurrent orally administered drugs like cyclosporine, midazolam, triazolam and calcium channel blockers such as felodipine and nisoldipine led to an increased serum concentration of these drugs due to CYP inhibition by phytochemical contents of the grapefruit juice [27]. Furthermore, researchers have suggested that the flavonoid content of grapefruit juice could have been exerting the inhibitory effects on CYP enzymes [28]. It is interesting to note that these inhibitory actions were absent with flavonoids found in oranges and other common fruits. The shortcoming in the commercial application of these flavonoids as bioavailability enhancers and/or GYP inhibitors is their ability to exert various physiological actions including their antioxidant activity [29, 30]. They are largely regarded as herbal extracted chemicals with non-uniform standards and are not approved for commercial pharmaceutical use by the US FDA, though they are commercially available.

There is therefore a need for new methods for inhibiting or reducing CYP3A4 metabolism of pharmaceutically active compounds.

### SUMMARY OF THE INVENTION

According to a first embodiment of the invention, there is provided a pharmaceutical composition for oral administration comprising:
a pharmaceutically active agent; and
an inhibitor of cytochrome P450 3A4 (CYP3A4) selected from the group consisting of polyethylene glycol), polyamine, poly(methyl methacrylate) and derivatives thereof;
wherein the inhibitor is present in an amount which is effective to substantially inhibit the pharmaceutically active agent from being pre-systemically metabolised when the composition is administered to a subject, resulting in a greater bioavailability of the pharmaceutically active agent than had the inhibitor not been present.

The poly(ethylene glycol) derivative may be selected from the group consisting of methoxy poly(ethylene glycol) having a molecular weight in the range of about 500 to about 10 000 g/mol, aminated poly(ethylene glycol) having a molecular weight in the range of about 500 to about 10 000 g/mol, 0-(2-aminoethyl)-β-methoxy poly(ethylene glycol) having a molecular weight of about 7500 g/mol, polyoxyethylene glycol having a molecular weight in the range of about 500 to about 10000 g/mol, branched poly(ethylene glycol) having a molecular weight in the range of about 500 to about 25000 g/mol, 3-arm poly(ethylene glycol), 4-arm poly(ethylene glycol) having a molecular weight in the range of about 10 000 g/mol to about 20 000 g/mol and 8-arm-poly(ethylene glycol) having a molecular weight in the range of about 10 000 g/mol to about 40 000 g/mol.

The polyamine derivative may be selected from the group consisting of poly(L-lysine), poly(L-arginine), poly(L-alanine), poly(L-valine), poly(L-serine), poly(L-histidine), poly(L-isoleucine), poly(L-leucine), poly(L-glutamic acid), poly(L-glutamine) and poly(L-guanidine).

The pharmaceutically active agent may be a substrate for CYP3A4 metabolism, such as felodipine.

The pharmaceutically active agent may be provided in an amount which is less than a therapeutic dose when the pharmaceutically active agent is administered without the inhibitor, but which is therapeutically effective when administered with the inhibitor.

According to a second embodiment of the invention, there is provided a method of increasing the bioavailability of an orally-administered pharmaceutically active agent in a subject, the method comprising administering an inhibitor of cytochrome P450 3A4 (CYP3A4) selected from the group consisting of poly(ethylene glycol), polyamine, poly(methyl methacrylate) and derivatives thereof and the pharmaceutically active agent to the subject, wherein the inhibitor is present in an amount which is effective to substantially inhibit the pharmaceutically active agent from being pre-systemically metabolised in the subject, resulting in a greater bioavailability of the pharmaceutically active agent than had the inhibitor not been present.

According to a third embodiment of the invention, there is provided poly(ethylene glycol) or a derivative thereof for use in a method of inhibiting cytochrome P450 3A4 (CYP3A4) metabolism of a pharmaceutically active agent in an animal or human.

According to a fourth embodiment of the invention, there is provided a polyamine or a derivative thereof for use in a method of inhibiting cytochrome P450 3A4 (CYP3A4) metabolism of a pharmaceutically active agent in an animal or human.

According to a fifth embodiment of the invention, there is provided a poly(methyl methacrylate) or a derivative thereof for use in a method of inhibiting cytochrome P450 3A4 (CYP3A4) metabolism of a pharmaceutically active agent in an animal or human.

According to a sixth embodiment of the invention, there is provided the use of a cytochrome P450 3A4 (CYP3A4) inhibitor in a method of making a medicament for use in a method of treating a human or animal, the medicament comprising an effective amount of the CYP3A4 inhibitor to prevent or reduce CYP3A4 metabolism of a pharmaceutically active agent when administered to the human or animal.

### BRIEF DESCRIPTION OF THE FIGURES

FIG. 1 shows the amino acid sequence of CYP3A4 isozyme (SEQ ID NO: 1);

FIG. 2 shows a three dimensional amino acid sequence of CYP 3A4 as shown by Hyperchem 7.5 professional software, revealing a) the structural morphology, b) the manipulable heme substructure, c) the expanded view of the C-terminal and d) the expanded view of the N-terminal;

FIG. 3 shows UPLC chromatograms of a) felodipine (5.037 minutes) and the appearance of its metabolite (3.542 minutes), and b) phenacetin (1.672 minutes) and the appearance of paracetamol, its metabolite (1.672 minutes);

FIG. 4 shows a non-linear regression profiling the felodipine concentration against the rate of metabolism by HLM;

FIG. 5 shows the inhibitory effects of flavonoids and verapamil on CYP3A4-dependent felodipine metabolism;

FIG. 6 shows a portion of CYP3A4 showing verapamil in the heme region;

FIG. 7 shows graphical models of the structural details of a) the bioreactive stations in CYP3A4 enhancing biomimetic reaction prediction showing protein fibril, b) a closer look at its coiling and c) tertiary structural domain depicting the bioresponsive domain for biomimetic activity;

FIG. 8 shows the inhibitory effects of 4-arm-PEG 10 000, 4-arm-PEG 20 000, 8-arm-PEG, MPEG-NH2, poly-L-lysine and poly(methyl methacrylate) on CYP3A4-catalyzed felodipine metabolism;

FIG. 9 shows a bar chart comparing the potencies of the investigated modelled modulators: A) verapamil, B) naringin, C) naringenin, D) quercetin, E) 4-arm-PEG 10000, F) 4-arm-PEG 20000, G) 8-arm-PEG, H) poly L-lysine, I) poly(methyl methacrylate) and J) MPEG-NH₂; and

FIG. 10 shows a bar chart comparing the IC₅₀ values of the modelled modulators in CYP3A4-expressed HLM and HIM: A) 4-arm-PEG, B) 4-arm-PEG 20000, C) 8-arm-PEG, D) poly-L-lysine, E) poly(methyl methacrylate) and F) MPEG-NH₂.

### DETAILED DESCRIPTION OF THE INVENTION

A pharmaceutical combination, dosage form or composition for oral administration of a pharmaceutically active agent to a subject is described herein, which includes the pharmaceutically active agent and an inhibitor of cytochrome P450 3A4 (CYP3A4). Administration of the inhibitor to a subject, whether prior to, concomitantly with, or after administration of the pharmaceutically active agent, inhibits or reduces pre-systemic degradation of the pharmaceutically active agent by CYP3A4. This results in an increase in the bioavailability of the pharmaceutically active agent. As a result, the pharmaceutically active agent may be more effective or may be therapeutically effective at a lower dose than had it not been administered with the inhibitor (which could also result in fewer side effects or a reduction in the cost of the therapy).

The inhibitor can be poly(ethylene glycol) or a derivative thereof, such as of methoxy poly(ethylene glycol) having a molecular weight in the range of about 500 to about 10 000 g/mol, aminated poly(ethylene glycol) having a molecular weight in the range of about 500 to about 10 000 g/mol, O-(2-aminoethyl)-O-methoxy poly(ethylene glycol) having a molecular weight of about 7500 g/mol, polyoxyethylene glycol having a molecular weight in the range of about 500 to about 10000 g/mol, branched poly(ethylene glycol) having a molecular weight in the range of about 500 to about 25000 g/mol, 3-arm poly(ethylene glycol), 4-arm poly(ethylene glycol) having a molecular weight in the range of about 10 000 to about 20 000 g/mol or 8-arm-poly(ethylene glycol) having a molecular weight in the range of about 10 000 g/mol to about 40 000 g/mol, and more particularly in the range of about 10 000 to 20 000 g/mol.

Alternatively, the inhibitor can be a polyamine, such as poly(L-lysine), poly(L-arginine), poly(L-alanine), poly(L-valine), poly(L-serine), poly(L-histidine), poly(L-isoleucine), poly(L-leucine), poly(L-glutamic acid), poly(L-glutamine) or poly(L-guanidine).

The inhibitor can also be poly(methyl methacrylate), polyvinyl acetate, polyacrylate, poly(lactic-co-glycolic acid) or derivatives thereof.

The pharmaceutically active agent can be a substrate for CYP3A4 metabolism, many of which are well-documented. These include immunosuppressants such as cyclosporins, tacrolimus and sirolimus; chemotherapeutics such as docetaxel, tamoxifen, paclitaxel, cyclophosphamide, doxorubicin, erlotinib, etoposide, ifosfamide, teniposide, vinblastine, vincristine, vindesine, imatinib, irinotecan, sorafenib, sunitinib, temsirolimus, anastrazole and gefitinib; anti-fungals such as ketoconazole and itraconazole; macrolides such as clarithromycin, erythromycin and telithromycin; tricyclic depressants such as amitriptyline, clomipramine and imipramine; selective serotonin reuptake inhibitors (SSRIs) such as citalopram, norfluoxetine and sertraline; general antidepressants such as mirtazapine, nefazodone, reboxetine, venlafaxine, and trazodone; anxiolytics such as buspirone; anti-psychotics such as haloperidol, aripiprazole, risperidone, ziprasidone, pimozide and quetiapine; opiates such as alfentanil, codeine, fentanyl, methadone and levacetylmethadol; benzodiazepines such as alprazolam, midazolam, triazolam and diazepam, hypnotics such as zopiclone, zaleplon and zolpidem; acetylcholinesterase inhibitors such as donepezil; statins such as atorvastatin, lovastatin, simvastatin and cerivastatin; calcium channel blockers such as diltiazem, felodipine, nifedipine, verapamil, amlodipine, lercanidipine, nitrendipine and nisoldipine; anti-arrhythmics such as amiodarone and quinidine; PDE5 inhibitors such as sildenafil and tadalafil; vaso-dialators such as kinins; sex hormones such as finasteride, estradiol, progesterone, ethinylestradiol, testosterone and toremifene; H1-receptor antagonists such as terfenadine, astermizole, chlorphenamine, indinavir, ritonavir, saquinavir and nelfinavir; non-nucleoside reverse transcriptase inhibitors such as nevirapine; glucocorticoids such as budesonide, hydrocortisone and dexamethasone; 5-HT4 receptor agonists such as cisapride; antiemetics such as aprepitant and loperamide; stimulants such as caffeine and cocaine; phosphodiesterase inhibitors such as cilostazol; anti-tussives such as dextromethorphan; anti-dopaminergics such as domperidone; aldosterone antagonists such as eplerenone; local anaesthetics such as lidocaine; 5-HT3 receptor agonists such as ondansetron; beta-blockers such as propranodol; beta-antagonists such as salmeterol; anticoagulants such as warfarin; antiplatelets such as clopidogrel; proton pump inhibitors such as esomeprazole; antidiabetics such as nateglinide; and anti-leprosy compounds such as dapsone.

The inhibitor can be conjugated with flavonoids or furanocoumarins found in grapefruit juice.

Advancements in computer-based computational modelling have opened the possibility of exploring the approach in predicting the natural and/or biological activity of various compounds when specific sets of input parameters are provided. Computational modeling is able to simulate natural and/or biological activity and can be employed to predict, for example, the relationship between xenobiotics and biological enzymes [31]. Understanding the complex nature of biochemical and/or pharmacological reactions at the cellular level has been made possible through computational molecular and structural rationalisation techniques [32, 33]. Quantitative structure-activity relationships (QSAR) of CYP substrates, their pharmacophoric units and the distinct amino acid sequence, molecular binding sites and overlapping substrate specificity of CYP are some of the inputs required to understand the biochemical basis of enzymatic reactions, predictability of outcomes on parameter variation and a deeper understanding of cellular reactions. Initial studies have reported successful application of two-dimensional and three-dimensional QSAR, pharmacophoric mapping and ligand-based computational modeling in predicting the affinity of structurally diverse compounds to CYP 2C9 and other CYP classes [34-39].

In order to generate potentially suitable CYP3A4 inhibitors that would improve bioavailability of an active pharmaceutical compound, computer modelling of CYP3A4 was conducted as described below.

Furthermore, in vitro studies of the metabolism of the pharmaceutically active compound and substrate of CYP3A4, felodipine, were conducted in Human Liver Microsomes (HLM) and were optimized yielding a typical Michaelis-Menten's plot from where the Kₘ and Vₘₐₓ values were estimated through non-linear regression. Naringin, naringenin and quercetin were incubated along with felodipine at the determined Kₘ value in HLM. The inhibitory action of flavonoids present in grapefruit juice (naringin, naringenin and quercetin) on CYP was verified using felodipine, a typical CYP 3A4 substrate, and verapamil, a known inhibitor as a control. The parameters involved in flavonoid-CYP reactions were employed in computational modeling to generate pharmaceutically acceptable pre-systemic enzymatic modulators which were tested for CYP inhibition employing Human Liver Microsomes (HLM) and Human Intestinal Microsomes (HIM). The positive inhibitory potencies obtained were determined and compared with those of flavonoids and verapamil.

Comparing results with those obtained with verapamil, a known CYP3A4 inhibitor, all three flavonoids inhibited felodipine metabolism. Through a detailed study of the QSAR of these flavonoids, their binding properties with CYP3A4, and the amino acid sequence and binding affinity of CYP3A4, computational modelling software was employed to identify pharmaceutically acceptable and commercially available polymers that can potentially be used as pre-systemic enzymatic modulators based on activity prediction and computational biomimetism. The modelled compounds were incubated with felodipine in both HLM and HIM mixtures in an approach similar to the flavonoids.

The results showed that, of the modelled polymers investigated, 8-arm-poly(ethylene glycol) (M_{W}=10000 g/mol.), O-(2-aminoethyl)-O-methyl poly(ethylene glycol), 4-arm-poly(ethylene glycol) (M_{W}=10000 g/mol.), poly L-lysine and 4-arm-poly(ethylene glycol) (M_{W}=20000 g/mol.) had inhibited the metabolism of felodipine with estimated 10₅₀ values of 7.22, 13.72, 16.28, 23.79, 29.68 and 30.0 μM in HLM and 5.78, 21.34, 15.92, 45.18, 19.20 and 41.03 μM in HIM, respectively. These novel computationally modelled pre-systemic enzymatic modulators that inhibited drug metabolism can therefore be employed as a strategy applicable in drug delivery for enhancing the oral bioavailability of various classes of active pharmaceutical compositions that are susceptible to CYP metabolism.

The invention will now be described in more detail by way of the following non-limiting examples.

### EXAMPLES

### Materials and Methods

### Materials

Pooled mixed gender human liver microsomes (HLM) expressing CYP3A4, CYP2C9, CYP4A11, CYP4F2, CYP2E1 and CYP2A6 were purchased from BD Biosciences (Pty) Ltd (Woburn, Mass., USA) and stored at −70° C. until used. Supply information indicated that microsomes were prepared from donor human livers (16 male, 14 female; 26 caucasians, 2 African-American and 2 Hispanics; age range 24-78 years; non-smokers, non-drinkers and non-liver related cause of death with no significant medical history). BD Biosciences also supplied pooled human intestinal microsomes (HIM) expressing CYP3A4, CYP2C9, CYP2J2, CYP4F12, UDT-glucuronosyl transferase and carboxylesterase prepared from matured enterocytes of both duodenum and jejunum sections of 5 donors (1 Male, 4 Female) with non-enteric related pathology as a cause of death. Methoxy poly(ethylene glycol) (M_{W}=5000 g/mol. [MPEG 5000] and 10000 g/mol. [MPEG 10000]), poly(ethylene glycol) (PEG, M_{W}=2000 g/mol. [PEG 2000] and 5000 g/mol. [PEG 5000]), 4-arm-poly(ethylene glycol) ([4-arm-PEG 10000] (M_{W}=10000 g/mol.) and [4-arm-PEG 20000] (M_{W}=20000 g/mol.)) and 8-arm-poly(ethylene glycol) (8-arm-PEG) (M_{W}=10000 g/mol.) were purchased from Jenkem Technology (Pty) Ltd (Beijing, China). Felodipine, verapamil and loperamide were purchased from Merck Chemicals (Pty) Ltd (Darmstadt, Germany). O-(2-aminoethyl)-O-methoxy poly(ethylene glycol) (M_{W}=7500 g/mol.) (MPEG-NH₂), quercetin, naringin naringenin, D-glucose 6-phosphate monosodium (G6P), glucose 6-phosphate dehydrogenase (G6PDH), poly L-lysine, poly(methyl methacrylate), poly(phenylalanine) and nicotinamide adenine dinucleotide phosphate (NADPH, reduced form) were obtained from Sigma-Aldrich (Pty) Ltd (St Louis, Mo., USA). All other chemicals used were of analytical grade and commercially available.

### Development of a UPLC Method for Felodipine Analysis

A method of quantitative determination of felodipine was developed using highly sensitive Ultra Performance Liquid Chromatography (UPLC). Standard curves were obtained with isocratic baseline separation of felodipine and loperamide using UPLC technology (Waters® Aquity UPLC™ System, MA, USA) comprising a binary solvent and a sample manager; a BEH C₁₈ column (1.7 μm; 2.1×50 mm) and a PDA detector set at 200 nm. Felodipine spiking solutions were prepared ranging from 31.25-1000 μmol/L. The mobile phase comprised 0.025M potassium dihydrogen phosphate buffer (pH 2.5) and acetonitrile (50:50) with a flow rate of 0.2 mL/min (7000 psi, delta<20) while 1.7 μL equal volume of sample and 50 μmol/L loperamide as an internal standard was injected into the column at 25° C. Loperamide eluted at 1.48±0.02 minutes while felodipine eluted at 5.1±0.02 minutes. Complete separation of the sample and the internal standard were confirmed by 3D chromatographic separation.

### Optimization of In Vitro Metabolism of Felodipine in HLM Incubations

### Preparation of Co-Factor and NADP-Regenerating Solutions

Co-factor concentrates containing 400 mg each of reduced NADP⁺ and G6P, and 266 mg of magnesium chloride pentahydrate were prepared in 20 mL deionised water and stored at −20° C. until use. G6PDH (40 U/mL) was prepared in 5 mM sodium citrate solution and stored at −20° C. until use. The NADP-regenerating system (NRS) comprised 1304 G6PDH, 6504 co-factor stock solution and was made up to 4.42 mL with 1.3 mL 0.5M phosphate buffer, pH 7.4 and deionised water. The preparation of NRS was completed right before use. It was made such that on addition to the HLM incubation mixture, it contained 2.6 mM NADP', 6.7 mM G6P, 6.6 magnesium chloride and 0.8 U/ml G6PDH.

### Preparation of Substrate and Microsomal Dilutions

Substrate (felodipine) solutions were prepared over a range of 0.01-100.0 mM in acetonitrile. Frozen HLM (−70° C.) was thawed by placing under cold running water and 5004 (5 mg/mL proteins) measured with a micropipette was diluted in 0.5M phosphate buffer (pH 7.4) to produce a final protein concentration of 0.5 mg/mL. This was kept on ice until use.

### In Vitro Metabolism of Felodipine in HLM Incubation

In 24-well plates, 5 μL felodipine solutions in duplicate were pre-warmed with 2504 0.5 mg/mL HLM for 5 minutes in a shaking orbital incubator (100 rpm; 37° C.) followed by the addition of 254 μL NRS enzymatic metabolic reactions and a further incubation for 15 minutes. The reaction was halted by a stop solution made of cooled acetonitrile (−20° C.).

Analysis of Substrate and Metabolite after Metabolic Reactions

The 24-well plates were transferred to a refrigerated centrifuge (Xiang Yi L-535R™ Centrifuge, Changsha, China) and the incubation mixtures centrifuged at 4° C. at 4000 g for 30 minutes to precipitate the microsomal proteins. Supernatants were filtered through 0.220 filters and analysed by the UPLC method developed in order to quantify the extent of felodipine metabolism. The rate of metabolism, determined as the rate of disappearance of substrate, was profiled against the substrate concentration to determine the enzyme kinetic parameters.

### Inhibition of CYP3A4-Catalysed Metabolism of Felodipine

Felodipine at its determined Kₘ was incubated in HLM as described above except for the addition of inhibitors. Verapamil, naringin, naringenin and quercetin were prepared in solutions at 6 different concentrations ranging from 30-1000 μM. Using a multi-channel pipette, 10 μL of each test solution was added to 250 μL diluted HLM solution (0.5 mg/mL) in duplicate in a 24-well plate and pre-warmed for 5 minutes in a shaking incubator (100 rpm; 37° C.). With the addition of NRS, microsomal activity was initiated and the plate incubated for 10 minutes followed by addition of felodipine into each well and a further incubation for 10 minutes. The incubation time of 10 minutes was selected such that approximately 50% of the felodipine was metabolized. Supernatants were analysed using the developed UPLC method. To determine the quantity of felodipine that was metabolised, control samples incubated with 0.05 mg/mL microsomal proteins without co-factors and NRS were subjected to a similar procedure. Another control was incubated without inhibitors to allow for maximum metabolism. All the tested inhibitors investigated are highly soluble in water except poly(methyl methacrylate) and the flavonoids. Acetonitrile is the preferred solvent for poorly water soluble compounds in in vitro determinations involving subcellular fractions. At higher concentrations, the effect of acetonitrile on enzymatic activity could be significant. It is therefore recommended that not >1% acetonitrile should be included in the incubation mixture [40]. Poly(methyl methacrylate), naringin, naringenin and quercetin were dissolved in acetonitrile. The final concentration of acetonitrile in the incubation mixture was 1%.

Computational Modeling of Bio-Modulators and Investigation of their Effects on In Vitro Felodipine Metabolism

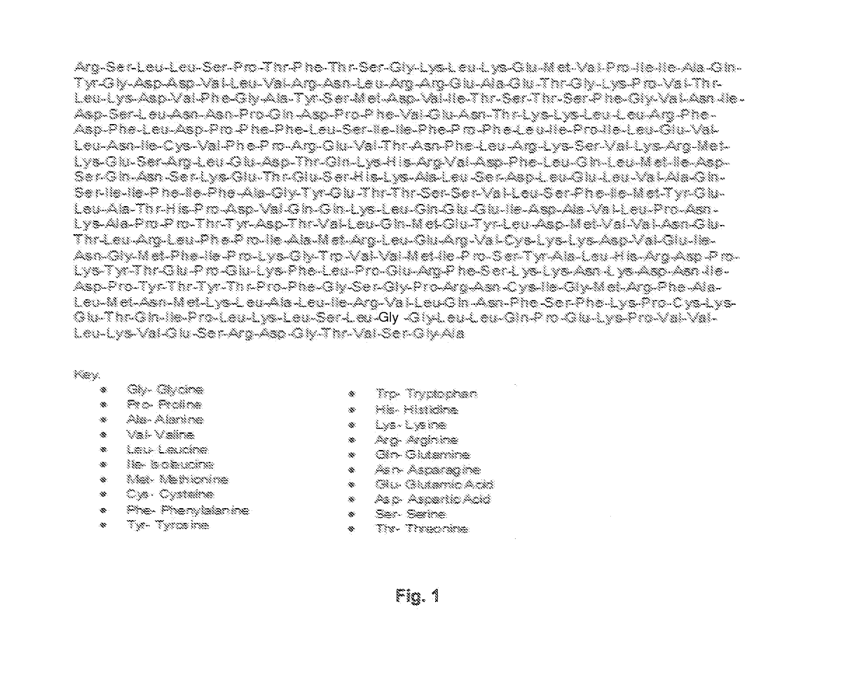

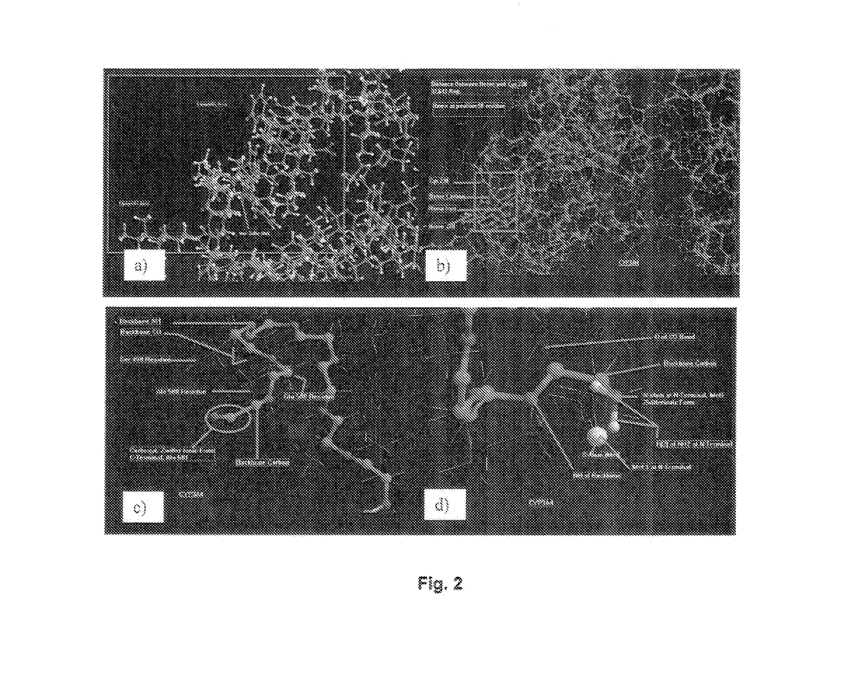

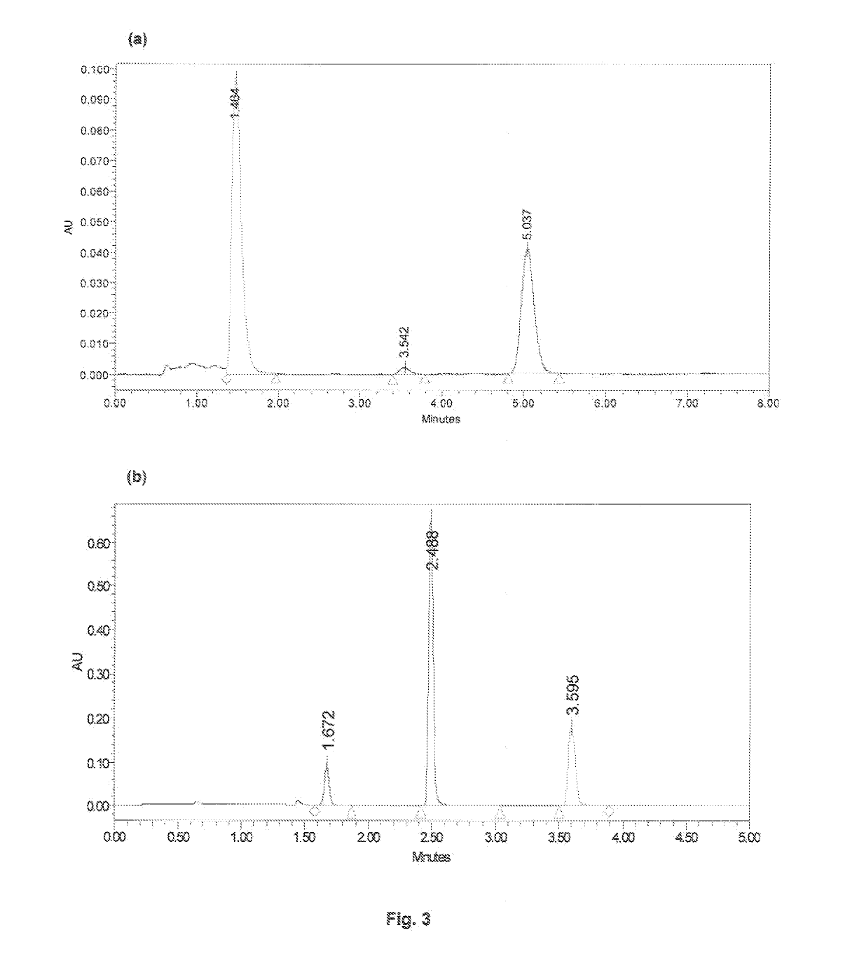

As an example, a computational comparative study of the structural and three-dimensional amino acid sequence of CYP3A4 (FIGS. 1 and 2) was conducted. FIG. 1 shows the amino acid sequence of CYP3A4 isozyme (SEQ ID NO: 1) [41]. The structural properties including the QSAR of flavonoids and verapamil were also modelled. Computational modelling was conducted using Hyperchem 7.5 professional computational modelling software on a non-silicon graphics system (HyperCube Inc. Gainesville, Fla., USA) (FIG. 3). The results were compared and analyzed.

The template derived from the known substrate felodipine was step-wise modified as a single variant within the structure taking into consideration the overall electronegativity/total charge density, dipole moment, bond length, bond angle, stereo-orientation and effective geometry. This provided a deeper molecular understanding of substrate specificity, binding affinity and manipulability of CYP3A4. The most stable forms of the resulting compounds were determined by estimations of the hydration energy and the energy of conformation. Polymer conjugation with known CYP3A4 inhibitors including high molecular mass flavonoids and furanocoumarins based on multi-site reactivity of the CYP variants was conducted.

The 3D modelling of the polypeptide structure demonstrated the manoeuvrability of the CYP3A4 polypeptide chain for the active site (heme substructure, located at Cys 58 residue). The constantly interchanging conformation of the protein chain due to physiological conditions inherent or induced by the xenobiotic factors and entities as well as the distance mapping of the active (heme) e) site, the different locations of the polypeptide chains and the proximity of amino acid residues by way of their conformation variability also confirmed this. The computational simulation was conducted with a distance maintained in the range of 2-25A mapped at 3 different locations from the N-terminal, middle of the chain and the C-terminal ending of the polypeptide chain suggesting the approach for the incoming molecule to the active site which in its conformation of binding and molecular volume would reach the biochemically active site. The proximity of other sulphur containing residues, especially from the methionine residues, may be at a complementary location to act as a scavenger (the S atom can act as a scavenger due to its high valency and can therefore readily form bonds or participate in electrostatic interactions with other atoms). Although the Cys 58 placement of the heme substructure is hypothetical, the link of the heme substructure to any other cystine residue would not have changed the activity except for a negligible change in the resulting loop which itself does not change the activity and approach of the chain and any xenobiotic entity attached to it for its interaction by detoxification via biochemical conversion.

It was therefore reasonable to propose that the very versatile activity of the CYP3A4, a polymorph of the CYP, is dependent on numerous factors. The fact that a range of molecules of different molecular masses, sizes, volumes and charges as well as diverse classes of compounds are substrates and inhibitors suggested a flexible approach to the bioactivity mechanism involved in interaction with CYP3A4 and its substrates. Thus, the binding to the chain of the molecules by both substrates and inhibitors is complementary and conjugative to the polypeptide chains of the active site. Computational simulation of bioactivity was thus used to predict and generate biodegradable, biocompatible natural and synthetic polymers and their conjugates capable of interacting with the CYP3A4 active domain.

### Analysis of the Inhibitory Effects of the Modelled Pre-Systemic Enzymatic Modulators on Felodipine Metabolism

Polymers including derivatives of poly(ethylene glycol), polyacrylates, polyoxyethelyne and polypeptides were sourced, prepared in a 0.01-100 mg/mL concentration range and incubated with felodipine separately in HLM and HIM solutions in a method similar to those employed with the flavonoids and verapamil. Their inhibitory effects were investigated on microsomal metabolism of felodipine. The potencies of the inhibitors were measured as their IC₅₀ values determined through non-linear regression of the percentage inhibition of felodipine metabolism against inhibitor concentration. The total quantity of felodipine metabolized within the incubation period was determined from control incubations without inhibition to be 32.97 μM. The concentration of felodipine in incubation mixtures containing the various inhibitors and the modelled pre-systemic enzymatic modulators was determined and the extent of metabolism was measured as percentage of control. The percentage inhibition observed was profiled against the concentration of inhibitors added to the incubation mixture. In each case a non-linear regression with a fit curve (R²≧0.99) and a regression equation were used to determine inhibitor concentration (IC₅₀) responsible for 50% substrate metabolism inhibition.

### Results and Discussion

### Chromatographic Separation and Quantitative Determination of Felodipine

Felodipine was eluted after 5.037±0.02 minutes (FIG. 3a). The assay method yielded a linear calibration curve through the origin with the relationship y=0.0158x where the ‘x’ and ‘y’ variables represented felodipine concentration and the ratio of the areas under the curve (AUC's) respectively. Assay method validation analysis revealed satisfactory intra- and inter-day precision and accuracy (R²=0.99). At the retention time and wavelength used, no interfering peaks were seen as observed through three-dimensional chromatographical analysis.

### Determination of Enzyme Kinetic Parameters of In Vitro Felodipine Metabolism

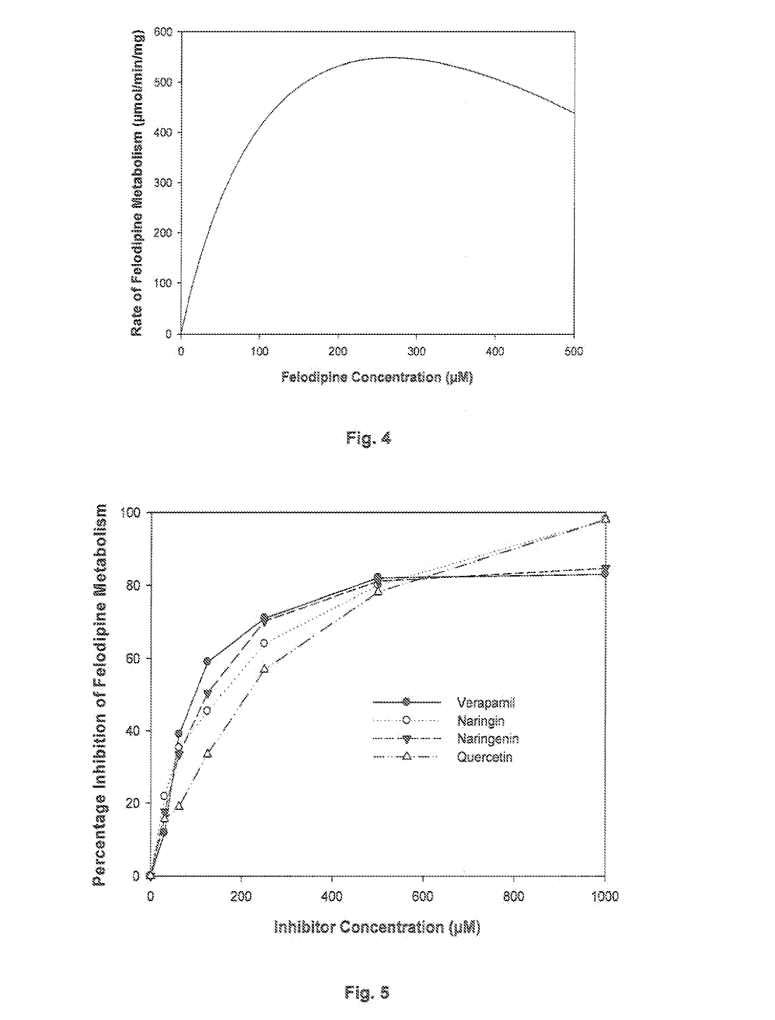

The rate of disappearance of felodipine from the incubation mixture as observed from the results obtained from UPLC analysis was used as evidence of enzymatic activity. By comparing the areas under the curve (AUC) of felodipine in the metabolized mixture with the control, the difference represents the quantity of substrate metabolized. The rate of metabolism was profiled against substrate concentration, a non-linear regression (R²=0.99) which yielded a Michaelis-Menten curve (FIG. 4). The Vₘₐₓ, (543.1 nmoL/min/mgHLM) and Kₘ (49.413 μM) were estimated. In a separate determination, the rate of formation of paracetamol by CYP1A2-mediated O-de-ethylation of phenacetin was determined (FIG. 3b). Similar results were obtained when the rate of phenacetin metabolism and rate of paracetamol formation were used as evidence of enzymatic action.

There is a direct correlation between in vitro and in vivo metabolism of xenobiotics by HLM and HIM [42-44]. As a result of this, new drug candidates are usually subjected to in vitro microsomal reactions to determine their susceptibility to pre-systemic metabolism, mode of elimination from the body and interaction with other drugs.

Standardization of laboratory techniques is often necessary in order to obtain reliable and reproducible results, and to determine the best substrate concentrations for optimal metabolic activity. HLM are subcellular fractions of the liver containing CYP enzymes, flavin monooxygenases and UDP glucuronyl transferases. HLM therefore requires stringent and fastidious conditions for optimal in vitro activity. Incubation in buffer solutions (pH 7.4) at 37° C., with co-factor solutions and a NADP generating system has been demonstrated to enhance in vitro metabolic activity of HLM comparative to in vivo behaviour [45].

In most enzymatic reactions, the Michaelis-Menten principle holds. According to this postulation, the initial rate of enzymatic reactions is directly proportional to substrate concentration. There is a substrate concentration where reactions are maximal (Vₘₐₓ) and beyond which the rate of metabolite formation is independent of further increase in substrate concentration. The substrate concentration corresponding to half the Vₘₐₓ is referred to as the Michaelis-Menten constant (Kₘ).

An optimal metabolic concentration for felodipine was predetermined in accordance with the Michaelis-Menten principle. A Kₘ value corresponding to the addition of 54 of 5 mM felodipine to a total incubation mixture of 0.5 mL with 0.25 mg/mL microsomal protein concentration agrees with earlier determinations [28]. Felodipine was the choice substrate in this study because the initial observation of influence of grapefruit juice on oral bioavailability was in patients on felodipine therapy and concomitant grapefruit ingestion [28]. The predominant metabolic inactivation of the dihydropyridines (DHP) is the oxidation of the DHP ring to form the pharmacologically inactive pyridines (Scheme 1). This step is mediated by CYP3A4 [46]. The initial assumption, therefore, was the modulation of CYP3A4 by chemical constituents of grapefruit juice. This formed the basis for the computational modelling in this study.

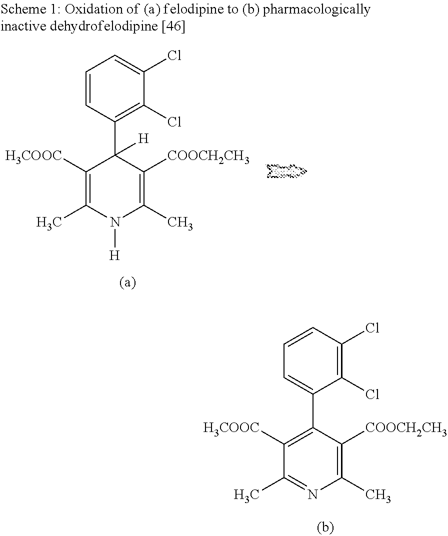

### Assessment of Inhibitory Effects of Flavonoids on Felodipine Metabolism in HLM Incubation Mixtures

The flavonoids co-incubated with felodipine in microsomal mixtures demonstrated an initial concentration-dependent inhibition of the rate and extent of felodipine metabolism. At higher concentrations, however, metabolism inhibition approached 100% (FIG. 5). This result is in agreement with earlier determinations suggesting that flavonoids and furanocoumarins present in grapefruit juice are responsible for the observed increase in plasma felodipine concentration following a concomitant ingestion of grapefruit juice by patients on felodipine therapy [27, 28]. Since binding affinity to receptor sites and subsequent interaction are structure-dependent, the parent structure of flavonoids thus becomes a potential precursor for structural and molecular modelling in this investigation.

### Computational Molecular and Structural Modelling of the Pre-Systemic Enzymatic Modulators

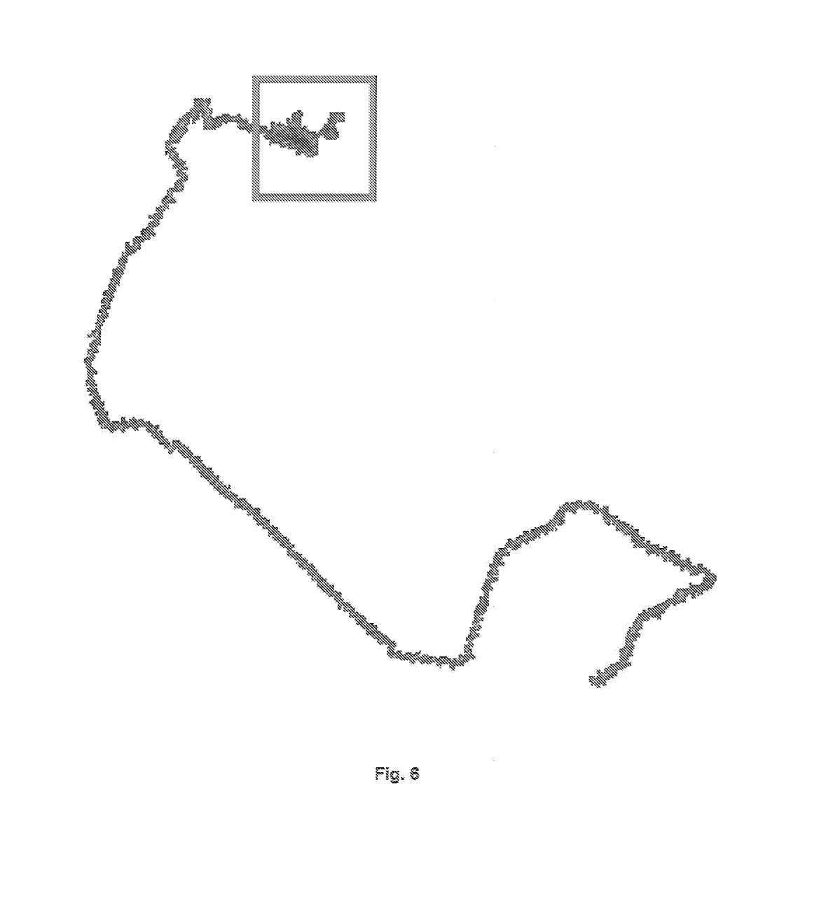

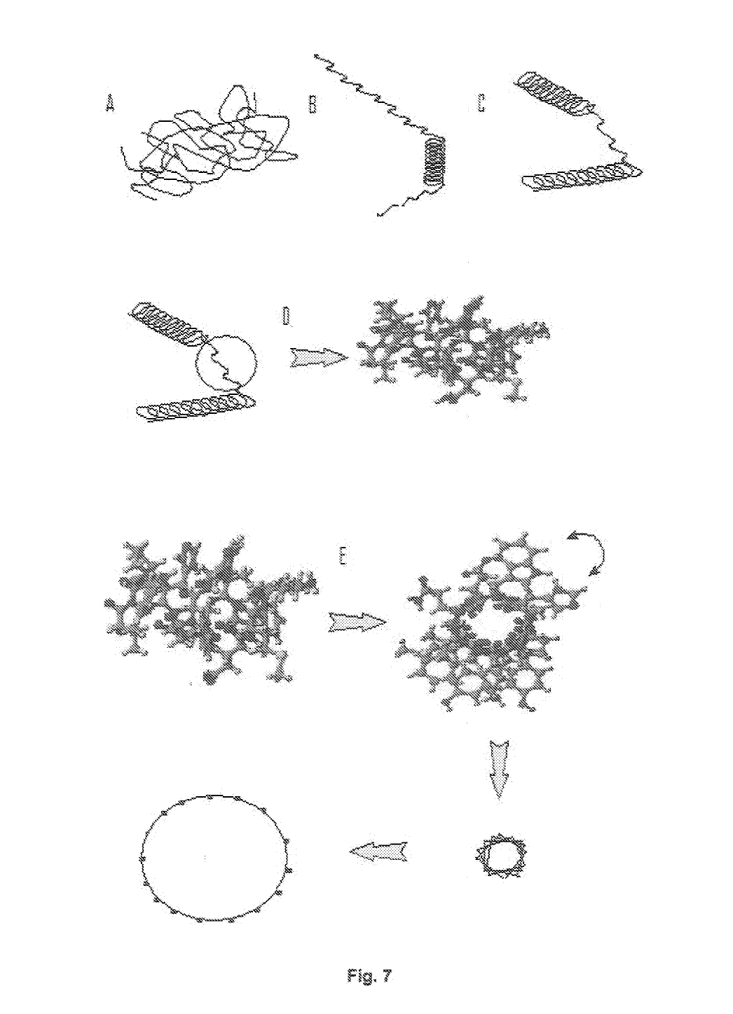

Computational modelling was used to elucidate structural and chemical requirements for CYP3A4 binding and activity. From the constituent structural topography for biomimetism (FIG. 2) the inter-structural cavity shape formation which may be responsible for activity was shaped by the presence of structural groups and their stereoelectronic factors present in specified reactive domain of the protein fibre which is taken as a clue for the formation of a nearly equivalent cavity with polymer structural groups. Stereoelectronic factors were also reliant by the surroundings. For a protein, the surrounding contribution is less significant due to the incompassive and ultra-advanced structural features of its molecule compared to polymers with less stereoelectronic and structural features at reactive domain or the interactive station. The binding of verapamil (FIG. 6) and flavonoids to the active site of CYP3A4 provides further understanding for computational modelling. Based on the computational biomolecular understanding of the biomodulatory mechanism of complex formation of flavonoids-CYP3A4 and verapamil-CYP3A4 complexes and subsequent action, computational biomimetism and simulation were used to predict and generate high molecular mass poly(ethylene glycol) based polymers and their derivatives, homopeptides, heteropeptides, polyoxyethylene and polyacrylate derivatives and conjugates (FIG. 7).

### Investigating the Effects of Modelled Compounds on Felodipine Metabolism

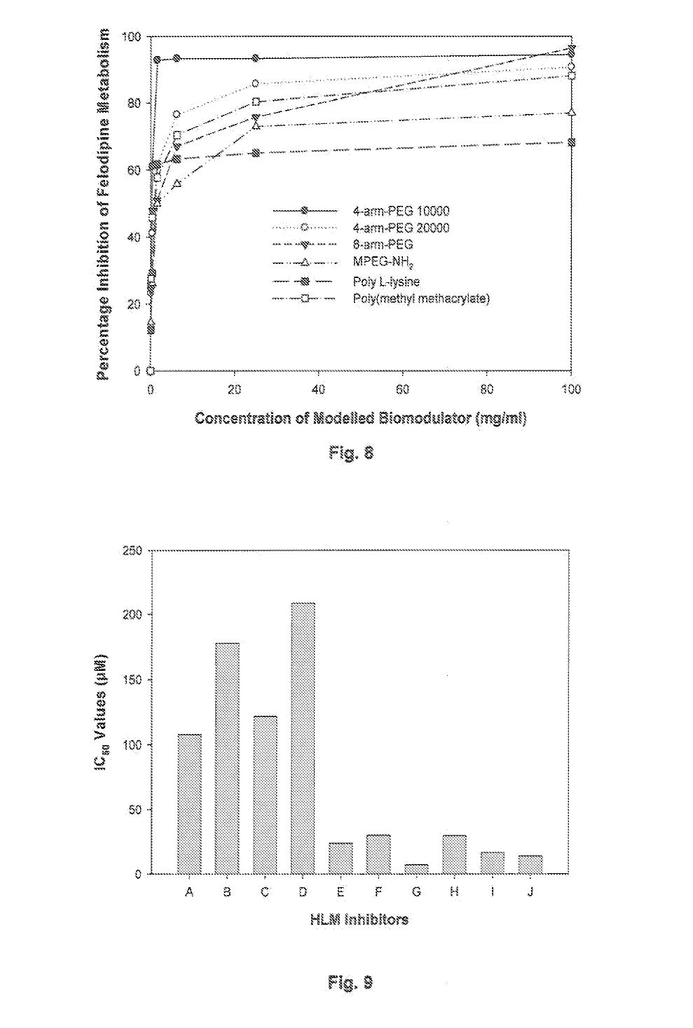

The modelled pre-systemic enzymatic modulators inhibited felodipine metabolism to varying degrees (FIG. 8). The potencies of some of the flavonoids and the modulators measured as their IC₅₀ values are compared in Table 1. Some of the modelled compounds (not shown) did not appear to have any significant influence on the rate of felodipine metabolism by HLM, suggesting that the mechanism of inhibition is complex and not entirely understood. PEG 2000, PEG 5000, methoxy PEG (M_{W}=1000 and 2000 g/mol.) and polyoxyethylene (M_{W}=7500 g/mol.) within the tested concentration range of 0.001-100 mg/mL did not have a significant effect on rate and extent of felodipine metabolism by HLM compared to the controls. The in vitro techniques suggest competitive receptor binding to the enzymes as a possible mechanism of felodipine inhibition. This is further demonstrated by the directly proportional relationship between the inhibitor concentrations and the initial extent of inhibition observed.

The ability of the modelled compounds to inhibit the metabolism of felodipine by HLM agrees with the earlier suggestion that the inhibitory effects of flavonoids are chemical structure-dependent [28] This opens up a reliable research route and mechanism in computational modelling. The ability of pegylated products to inhibit CYP-induced metabolism has also drawn attention to pharmaceutical grade polymers in the search of oral bioavailability enhancers. The results show a higher inhibitory potency for 8-arm-PEG than 4-arm-PEGs and higher potency of 4-arm-PEG 20000 than 4-arm-PEG 10000. This suggests that the higher the molecular mass of pegylated products, the more their affinity for CYP binding and possible interaction with the active domain. Although enzymatic catabolism has been reported to be responsible for high intestinal first pass effect of dietary proteins [47], amino acids have not been reported to inhibit human microsomal cytochromes. The ability of poly-L-lysine, for example, to inhibit CYP3A4-mediated metabolism of felodipine (as shown in FIGS. 8 and 9), however, suggests that amino acid derivatives offer hope in the search of oral bioavailability enhancers.

TABLE 1
Summary table comparing the inhibitory properties of
various HLM inhibitors and modelled biomodulators

| | | | | % Max. |
|---|---|---|---|---|
| | IC₅₀ | | Highest | [Inhibitory] |
| Inhibitor | (mg/mL) | IC₅₀ (μM) | inhibition | (μM) |
| Verapamil | 0.0530 | 107.88 | 83 | 500 |
| Naringin | 0.1032 | 177.81 | 98 | 1000 |
| Naringenin | 0.0332 | 121.97 | 95 | 500 |
| Quercetin | 0.0631 | 208.65 | 98 | 750 |
| 4-Arm-PEG 10000 | 0.2379 | 23.79 | 95 | 10000 |
| 4-Arm-PEG 20000 | 0.6000 | 30 | 91 | 5000 |
| 8-Arm-PEG 10000 | 0.0722 | 7.22 | 96 | 10000 |
| Poly L-lysine | 0.2226 | 29.68 | 69 | 1000 |
| Poly(methyl | 0.1628 | 16.28 | 88 | 26667 |
| methacrylate) 10000 | | | | |
| O-(2-aminoethyl)-O- | 0.1029 | 13.72 | 77 | 1000 |
| methoxy PEG 7500 | | | | |

### Comparative Inhibition of Felodipine Metabolism by Modelled Biomodulators in HLM and HIM

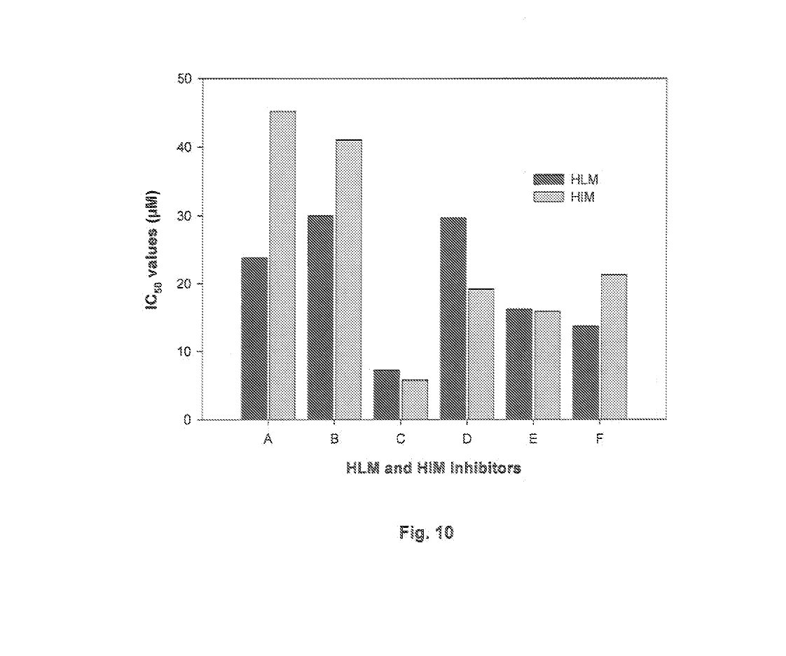

In vitro drug metabolism for in vivo correlation often employs HLM. Pre-systemic drug metabolism occurs principally in the intestinal wall and the liver. An HLM inhibitor may not necessarily have the same effect in the intestines although cytochromic morphology might be the same. If this is the case, prospective oral bioavailability must necessarily be absorbed for hepatic activity. An enzyme inhibitor active against intestinal CYP will prevent intestinal pre-systemic metabolism and enhance oral bioavailability. Thus the use of HIM was to investigate the effects of HLM inhibitors on intestinal metabolism for a more reliable extrapolation. The modelled modulators were incubated with felodipine at the predetermined Kₘ value with CYP3A4 expressed HIM. This was for in vivo correlational reliability. Prospective in vivo HLM modulators must be absorbed. It was reasoned that compounds active against HLM may not survive the physicochemical barriers necessary for hepatic absorption. The use of HIM estimates the potential utility of the modelled modulators that are pre-systemically degradable and whose potencies may be lost outside the gastrointestinal tract. From the results obtained, the modelled HLM inhibitors demonstrated inhibitory actions against HIM metabolism of felodipine (Tables 2 and 3, and FIG. 10). Thus, this confirms the potential utility of the modelled pre-systemic enzymatic modulators in oral drug delivery for enhancing the oral bioavailability of various classes of active pharmaceutical compositions.

TABLE 2
Inhibitory potencies of modelled biomodulators in HLM and HIM

| | IC₅₀ values in HLM | % Inhibition | % Inhibition |
|---|---|---|---|
| Modelled Modulators | (μM) | in HLM | in HIM |
| 4-arm-PEG 10000 | 23.79 | 50 | 33.90 |
| 4-arm-PEG 20000 | 30 | 50 | 48.69 |
| 8-arm-PEG 10000 | 7.22 | 50 | 71.03 |
| Poly-L-lysine | 29.68 | 50 | 67.45 |
| Poly(methyl | 16.28 | 50 | 54.51 |
| methacrylate) 10000 | | | |
| O-(2-aminoethyl)-O- | 13.72 | 50 | 36.16 |
| methoxy PEG 7500 | | | |

TABLE 3
IC₅₀ values of the modelled pre-systemic enzymatic modulators

| Modelled Biomodulators | IC₅₀ (μM) in HLM | IC₅₀ (μM) in HIM |
|---|---|---|
| 4-arm-PEG 10000 | 23.79 | 45.18 |
| 4-arm-PEG 20000 | 30 | 41.03 |
| 8-arm-PEG 10000 | 7.22 | 5.78 |
| Poly-L-lysine | 29.68 | 19.20 |
| Poly(methyl methacrylate) | 16.28 | 15.92 |
| 10000 | | |
| O-(2-aminoethyl)-O-methoxy | 13.72 | 21.34 |
| PEG 7500 | | |

### REFERENCES

1. Cross, D. M. and Bayliss, M. K. (2000). A commentary on the use of hepatocytes in drug metabolism studies during drug discovery and development. Drug Metabolism Reviews 32(2):219-240
2. Davila, J. C., Rodriguez, R. J., Melchert, R. B., and Acosta, D. (1998). Predictive value of in vitro model systems in toxicology. Annu. Rev. Pharmacol. Toxicol. 38:63-96
3. Ehrhardt, C. and Forbes, B. (2005). Human respiratory epithelial cell culture for drug delivery applications. European Journal of Pharmaceutics and Biopharmaceutics 60(2):193-205
4. Ekins, S., Ring, B. J., Grace, J., McRobie-Belle, D. J. and Wroghton, S. A. (2000). Present and future in vitro approaches for drug metabolism. Journal of Pharmacological and Toxicological Methods 44(1):313-324
5. Breedveld, P., Beijnen, J. H. and Schellens, J. H. M. (2005). Use of p-glycoprotein and BCRP inhibitors to improve oral bioavailability and CNS penetration of anticancer drugs. Trends in Pharmcological Sciences 27(1):17-24
6. Agoram, B., Woltosz, W. S. and Bolger, M. B. (2001). Predicting the impact of physiological and biochemical processes on oral drug bioavailability. Advanced Drug Delivery Reviews 50(1): S41-S67
7. Uwe, F. (2000). Induction of drug metabolising enzymes: pharmacokinetic and toxicological consequences in humans. Clinical Pharmacokinetics 38(6):493-504
8. Fang, X. and Xiao-yin, C. (2005). Effects of intestinal cytochrome P450 3A on phytochemical pre-systemic metabolism. Chinese Journal of Integrative Medicine 11(3):232-236
9. Zhang, Y. and Benet, L. Z. (2001): The gut as a barrier to drug absorption: combined role of cytochrome P450 3A and p-glycoprotein. Clinical Pharmacokinetics 40(3):159-168
10. Nebert, D. W. and Dalton, T. P. (2006). The role of cytochrome P450 enzymes in endogenous signalling pathways and environmental carcinogenesis. Nature Reviews Cancer 6:947-960
11. Guengerich, F. P. (2003). Cytochromes P450, drugs, and diseases. Molecular Interventions 3:194-204
12. Gonzalez, F. G. (2005). Role of cytochromes P450 in chemical toxicity and oxidative stress: studies with CYP2E1. Mutation Research 569(1-2):101-110
13. Handschin, C., Podvinec, M., Amherd, R., Looser, R., Ourlin, C. and Meyer, U. A. (2002). Cholesterol and bile acids regulate xenosensor signalling in drug-mediated induction of cytochromes P450. J. Biol. Chem. 277(33):29561-29567
14. Rendic, S. (2002). Summary of information on human CYP enzymes: human P450 metabolism data. Drug metabolism Reviews 34(1):83-448
15. Farid, N. A., Payne, C. D., Small, D. S., Winters, K. J., Ernest, C. S., Brandt, J. T., Darstein, C., Jakubowski, J. A. and Salazar, D. E. (2007). Cytochrome P450 3A inhibition by ketoconazole affects prasugrel and clopidogrel pharmacokinetics and pharmacodynamics differently. Clinical Pharmacology and Therapeutics 81:735-741
16. Cotreau, M. M., Moltke, L. L. and Greenblatt, D. (2005). The influence of age and sex on the clearance of cytochrome P450 3A substrates. Clinical Pharmacokinetics 44(1):33-60
17. Gorski, J. C., Vannaprasaht, S., Hamman, M. A., Ambrosius, W. T., Bruse, M. A., Haehner-Daniels, B and Hall, S. D. (2003). The effect of age, sex, and rifampin administration on intestinal and hepatic cytochrome P450 3A activity. Clinical Pharmacology and Therapeutics 74:275-287
18. Quintieri, L., Palatini, P., Nassi, A., Ruzza, P. and Floreani, M. (2007). Flavonoids diosmetin and luteolin inhibit midazolam metabolism by human liver microsomes and recombinant CYP 3A4 and CYP 3A5 enzymes. Biochemical Pharmacology 75:1426-1437
19. Dresser, G. K., Spence, J. D. and Bailey D. G. (2000). Pharmacokinetic-pharmacodynamic consequences and clinical relevance of cytochrome P450 3A4 inhibition. Clinical Pharmacokinetics 38(1):41-57
20. Obach, R. S., Walsky, R. L., Venkatakrishnan, K., Gaman, E. A., Houston, J. B. and Tremaine, L. M. (2006). The utility of in vitro cytochrome P450 inhibition data in the prediction of drug-drug interactions. Journal of Pharmacology and Experimental Therapeutics 316:336-348
21. Ohyama, K., Nakajima, M., Suzuki, M., Shimada, N., Yamazaki, H. and Yokoi, T. (2000). Inhibitory effects of amiodarone and its N-deethylated metabolite on human cytochrome P450 activities: prediction of in vivo drug interaction. Br J. Clin. Pharmacol. 49(3):244-253
22. Wang, R. W., Newton, D. J., Liu, N., Atkins, W. M. and Lu, A>Y>H>(2000). Human cytochrome P450 3A4: in vitro drug-drug interaction patterns are substrate dependent. Drug metabolism and Disposition 28(3):360-366
23. Masaoka, Y., Tanaka, Y. and Kataoke, M. et al., (2006). Site of absorption after oral administration: Assessment of membrane permeability and luminal concentration of drugs in each segment of gastrointestinal tract. Eur. J. Pharm. Sci., in press.
24. Majundar, S, and Mitra, A., (2006). Chemical modification and formulation approaches to elevated drug transport across cell membranes. Expert Opin. Drug Deliv. 3(4):511-527
25. Leonard, T. W., Lynch, J. and McKenna, M. J. et al., (2006). Promoting absorption of drugs in human medium chain fatty acid-based solid dosage forms. GIPET Expert. Opin. Drug Deliv., 3(5):685-692
26. Hamman, J. H., Enslin, G. M. and Kotze, A. F., (2005) Oral delivery of peptide drugs. Biodrugs, 19:165-177
27. Bailey, D. G., Arnold, J. M. O. and Spence, J. D., (1998), Grapefruit juice-drug interactions. Br J Clin Pharmacol, 46:101-110
28. Miniscalco, A., Lundahl, J., Regardh, C. G., Edgar, B. and Eriksson, U. G. (1992). Inhibition of dihydropyridine metabolism in rat and human liver microsomes by flavonoids found in grapefruit juice. The Journal of Pharmacology and Experimental Therapeutics 261 (3):1195-1199
29. Ohshima, H., Yoshie, Y, Auriol, S. and Gilibert, I. (1998). Antioxidant and pro-oxidant actions of flavonoids: effects on DNA damage induced by nitric oxide, peroxynitrite and nitroxyl anion. Free Radical Biology and Medicine 25(9):1057-1065
30. Haysteen, B. H. (2002). The biochemistry and medical significance of the flavonoids. Pharmacology and Therapeutics 96(2-3):67-202
31. Long, A. and Walker, J. D. (2003). Quantitative structure-activity relationship for predicting metabolism and modeling cytochrome P450 enzyme activities. Environmental Toxicology and Chemistry 22(8):1894-1899
32. Sibeko, B., Pillay, V., Choonara, Y. E., Khan, R. A., Modi, G. and lyuke, S. E. (2009). Computaional molecular modeling and structural rationalization for the design of drug-loaded PLLA/PVA biopolymeric membrane. Biomed. Mater. 4:1-10
33. Ironi, L. and Tentoni, S. (2003). A model based approach to the assessment of physicochemical properties of drug delivery materials. Comput. Chem. Eng. 27:803-12
34. Rao, S., Aoyama, R., Schrag, M., Trager, W. F., Rettie, A. and Jones, J. P. (2000). A refined 3-dimentional QSAR of cytochrome P450209: computational predictions of drug integration. J. Med. Chem. 43:2789-2796
35. Rieko, A. (2006). Computational models for predicting interactions with cytochrome P450 enzymes. Current Topics in Medicinal Chemistry 6(15):1609-1618
36. Herwaarden, A., Waterschool, R. A. B. and Schinkel, A. H. (2009). How important is intestinal cytochrome P450 3A metabolism. Trends in Pharmacological Sciences 30(5):223-227
37. Goosen, T. C., Cillie, D., Bailey, D. G., Yu, C., He, K., Hollenberg, P. F., Woster, P. M., Cohen, L. et al. (2004). Bergamottin contribution to the grapefruit juice-felodipine interaction and disposition in humans. Clinical Pharmacology and Therapeutics 76:607-617
38. Yeo, K. R. and Yeo, W. W. (2001). Inhibitory effects of verapamil and diltiazem on simvastatin metabolism in human. Br. J. Clin. Pharmacol. 51:461-470
39. Foster, B. C. (2001). An in vitro evaluation of human cytochrome P450 3A4 and p-glycoprotein inhibition by garlic. J. Pharm. Pharmaceut. Sci 4(2)176-184
40. Celsis In vitro Technologies (2009). In vitro CYPTMM-classTM Human Liver Microsomes. http://www.celsis.com/media/pdf/filelib/ps_InVitroCYPMclass.pdf accessed Aug. 31, 2009
41. http://drnelson.utmem.edu/human.blast.file.html accessed on Aug. 31, 2009.
42. Moltke, L. L., Greenblatt, D. J., Granda, B. W., Duan, S. X. et al (1999). Zolpidem metabolism in vitro: responsible cytochromes, chemical inhibitors, and in vivo correlations. Br. J. Clin. Pharmacol. 48(1):89-97
43. Martinez, C., Albert, C., Agundez, J. A. G., Herrero, E. et al (1999). Comparative in vitro and in vivo inhibition of cytochrome P450: CYP1A2, CYP2D6 and CYP3A by H2-receptor antagonists. Clinical Pharmacology and Therapeutics 65:369-376
44. Schmider, J., Von Moltke, L. L., Shader, R. I., Harmatz, J. S, and Greeblatt, J. (1999). Extrapolating in vitro data on drug metabolism to in vivo pharmacokinetics: evaluation of the pharmacokinetic interaction between amitriptyline and fluoxetin. Drug Metabolism Reviews 31(2):545-560
45. Yan, G. and Caldwell (2005). Evaluation of cytochrome P450 inhibition in human liver microsomes, From: Methods in Pharmacology and Toxicology, Optimization in Drug Discovery: In Vitro Methods (231-244)
46. Cardoza, R. M. and Amin, P. D. (2002). A stability indicating LC method for felodipine. Ournal of Pharmaceutical and Biomedical Analysis 27(5):711-718
47. Stoll, B., Henry, J., Reeds, P. and Yu, H. (1998). Catabolism dominates the first-pass intestinal metabolism of dietary essential amino acids in milk protein-fed piglets

1. A pharmaceutical composition for oral administration comprising:
a pharmaceutically active agent; and
an inhibitor of cytochrome P450 3A4 (CYP3A4) selected from the group consisting of poly(ethylene glycol), polyamine, poly(methyl methacrylate) and derivatives thereof;
wherein the inhibitor is present in an amount which is effective to substantially inhibit the pharmaceutically active agent from being pre-systemically metabolised when the composition is administered to a subject, resulting in a greater bioavailability of the pharmaceutically active agent than had the inhibitor not been present.

2. A pharmaceutical composition according to claim 1, wherein the inhibitor is poly(ethylene glycol) or a derivative thereof.

3. A pharmaceutical composition according to claim 2, wherein the inhibitor is selected from the group consisting of methoxy poly(ethylene glycol) having a molecular weight in the range of about 500 to about 10 000 g/mol, aminated poly(ethylene glycol) having a molecular weight in the range of about 500 to about 10 000 g/mol, 042-aminoethyl)-O-methoxy poly(ethylene glycol) having a molecular weight of about 7500 g/mol, polyoxyethylene glycol having a molecular weight in the range of about 500 to about 10000 g/mol, branched poly(ethylene glycol) having a molecular weight in the range of about 500 to about 25000 g/mol, 3-arm poly(ethylene glycol), 4-arm poly(ethylene glycol) having a molecular weight in the range of about 10 000 g/mol to about 20 000 g/mol and 8-arm-poly(ethylene glycol) having a molecular weight in the range of about 10 000 g/mol to about 20 000 g/mol.

4. A pharmaceutical composition according to claim 1, wherein the inhibitor is 8-arm-poly(ethylene glycol).

5. A pharmaceutical composition according to claim 2, wherein the inhibitor is a polyamine or derivative thereof.

6. A pharmaceutical composition according to claim 5, wherein the inhibitor is selected from the group consisting of poly(L-lysine), poly(L-arginine), poly(L-alanine), poly(L-valine), poly(L-serine), poly(L-histidine), poly(L-isoleucine), poly(L-leucine), poly(L-glutamic acid), poly(L-glutamine) and poly(L-guanidine).

7. A pharmaceutical composition according to claim 2, wherein the inhibitor is poly(methyl methacrylate).

8. A pharmaceutical composition according to claim 1, wherein the pharmaceutically active agent is a substrate for CYP3A4 metabolism.

9. A pharmaceutical composition according to claim 1, wherein the pharmaceutically active agent is felodipine.

10. A pharmaceutical composition according to claim 1, wherein the pharmaceutically active agent in the composition is provided in an amount which is less than a therapeutic dose when the pharmaceutically active agent is administered without the inhibitor, but is therapeutically effective when administered with the inhibitor.

11. A method of increasing the bioavailability of an orally-administered pharmaceutically active agent in a subject, the method comprising administering an inhibitor of cytochrome P450 3A4 (CYP3A4) selected from the group consisting of poly(ethylene glycol), polyamine, poly(methyl methacrylate) and derivatives thereof and the pharmaceutically active agent to the subject, wherein the inhibitor is present in an amount which is effective to substantially inhibit the pharmaceutically active agent from being pre-systemically metabolised in the subject, resulting in a greater bioavailability of the pharmaceutically active agent than had the inhibitor not been present.

12. A method according to claim 11, wherein the inhibitor is poly(ethylene glycol) or a derivative thereof.

13. A method according to claim 12, wherein the inhibitor is selected from the group consisting of methoxy poly(ethylene glycol) having a molecular weight in the range of about 500 to about 10 000 g/mol, aminated poly(ethylene glycol) having a molecular weight in the range of about 500 to about 10 000 g/mol, O-(2-aminoethyl)-O-methoxy poly(ethylene glycol) having a molecular weight of about 7500 g/mol, polyoxyethylene glycol having a molecular weight in the range of about 500 to about 10000 g/mol, branched poly(ethylene glycol) having a molecular weight in the range of about 500 to about 25000 g/mol, 3-arm poly(ethylene glycol), 4-arm poly(ethylene glycol) having a molecular weight in the range of about 10 000 g/mol to about 20 000 g/mol and 8-arm-poly(ethylene glycol) having a molecular weight in the range of about 10 000 g/mol to about 20 000 g/mol.

14. A method according to claim 13, wherein the inhibitor is 8-arm-poly(ethylene glycol).

15. A method according to claim 11, wherein the inhibitor is a polyamine or derivative thereof.

16. A method according to claim 15, wherein the inhibitor is selected from the group consisting of poly(L-lysine), poly(L-arginine), poly(L-alanine), poly(L-valine), poly(L-serine), poly(L-histidine), poly(L-isoleucine), poly(L-leucine), poly(L-glutamic acid), poly(L-glutamine) and poly(L-guanidine).

17. A method according to claim 11, wherein the inhibitor is poly(methyl methacrylate).

18. A method according to claim 11, wherein the pharmaceutically active agent is a substrate for CYP3A4 metabolism.

19. A method according to claim 11, wherein the pharmaceutically active agent is felodipine.

20. A method according to claim 11, wherein the pharmaceutically active agent is administered in an amount which would not be therapeutic when administered without the inhibitor, but is a therapeutic amount when administered with the inhibitor.

21. Poly(ethylene glycol) or derivative thereof for use in a method of inhibiting cytochrome P450 3A4 (CYP3A4) metabolism of a pharmaceutically active agent in an animal or human.

22. The poly(ethylene glycol) or derivative thereof according to claim 21, which is selected from the group consisting of methoxy poly(ethylene glycol) having a molecular weight in the range of about 500 to about 10 000 g/mol, aminated poly(ethylene glycol) having a molecular weight in the range of about 500 to about 10 000 g/mol, 042-aminoethyl)-O-methoxy poly(ethylene glycol) having a molecular weight of about 7500 g/mol, polyoxyethylene glycol having a molecular weight in the range of about 500 to about 10000 g/mol, branched poly(ethylene glycol) having a molecular weight in the range of about 500 to about 25000 g/mol, 3-arm poly(ethylene glycol), 4-arm poly(ethylene glycol) having a molecular weight in the range of about 10 000 g/mol to about 20 000 g/mol and 8-arm-poly(ethylene glycol) having a molecular weight in the range of about 10 000 g/mol to about 20 000 g/mol.

23. The poly(ethylene glycol) or derivative thereof according to claim 22, which is 8-arm-poly(ethylene glycol).

24. Polyamine or a derivative thereof for use in a method of inhibiting cytochrome P450 3A4 (CYP3A4) metabolism of a pharmaceutically active agent in an animal or human.

25. The polyamine or derivative thereof of claim 24, which is selected from the group consisting of poly(L-lysine), poly(L-arginine), poly(L-alanine), poly(L-valine), poly(L-serine), poly(L-histidine), poly(L-isoleucine), poly(L-leucine), poly(L-glutamic acid), poly(L-glutamine) and poly(L-guanidine).

26. Poly(methyl methacrylate) or a derivative thereof for use in a method of inhibiting cytochrome P450 3A4 (CYP3A4) metabolism of a pharmaceutically active agent in an animal or human.

27-37. (canceled)
